(19) United States
(12) Patent Application Publication
(10) Pub. No.: US 2005/0164260 A1
(43) Pub. Date: Jul. 28, 2005

(54) Rapid preparation of nucleic acids by enzymatic digestion

(75) Inventor: Fuqiang Chen, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Sigma-Aldrich Co., St. Louis, MO (US)

(21) Appl. No.: 11/027,150

(22) Filed: Dec. 30, 2004

Related U.S. Application Data
(60) Provisional application No. 60/533,624, filed on Dec. 30, 2003.

(51) Int. Cl. C12Q 1/68; C12N 1/08

(52) U.S. Cl. 435/6; 435/270

(57) ABSTRACT

The present invention provides methods, compositions and kits for isolating and purifying at least one nucleic acid directly from a biological sample, or for preparing a biological sample for subsequent isolation and purification of at least one nucleic acid, without removal of the biological sample's cell culture medium or cellular fluid.

### REFERENCE TO RELATED APPLICATIONS

This application claims the benefit of U.S. Provisional Application Ser. No. 60/533,624, filed Dec. 30, 2003, the entire contents of which are incorporated herein by reference.

### BACKGROUND OF THE INVENTION

Advances in nucleic acid applications, such as automated sequencing and drug screening, have increased the need for a quick, efficient, and cost-effective method for isolating and purifying nucleic acids such as DNA or RNA in all forms (linear and circular, including plasmids or vectors). To obtain sufficient copies of a DNA of interest, for example, a researcher places the DNA of interest into a vector or plasmid. The researcher then transforms the constructed plasmid into a cell, such as a bacterial cell (e.g., E. coli). The bacterial cell is then grown on a selective solid medium, such as agar, and through cell division, a colony of identical bacterial cells containing the constructed plasmid is developed. The researcher inoculates this colony into a liquid medium, such as LB broth, and allows the colony to multiply overnight.

The prevalent methods of plasmid preparation require (1) harvesting of cells from culture, usually by centrifugation to pellet the cells containing the plasmid, and, after centrifugation, (2) removal of the culture medium. After harvesting, an additional step is taken where the cells are reconstituted in a resuspension buffer by vortexing, shaking, or pipetting. These common steps do not automate well because they require additional cost, time and manipulation to carry out. Systems that attempt to automate these steps are extremely expensive. Only after harvesting cells, removing the culture medium and resuspending the cells are the cells finally lysed.

The art discloses that culture medium interferes with subsequent manipulation of isolated plasmids. For example, Sambrook, J., et al., Molecular Cloning, A Laboratory Manual, 2nd ed., Cold Spring Harbor Laboratory Press, 1989, at p. 1.31, states that “[w]hen workers make minipreps for the first time, the plasmid DNA sometimes is resistant to cleavage by restriction enzymes. Almost always, this problem occurs because insufficient care is taken to remove all of the supernatant fluid from the pellet of bacteria.”

Lysis is usually accomplished by utilizing base, detergent, enzymes and/or heat. Neutralization and subsequent lysate clearing steps are required when an alkaline lysis method is used to lyse cells. These methods require laborious and lengthy steps, which prevent developing a rapid method of preparing nucleic acids.

An alternative method for isolating DNA from a DNA source, e.g., blood, saliva, bacterial cultures, etc., involves lysing the DNA source with a combination of a proteolytic enzyme and a detergent followed by extraction of the mixture with an organic solvent, e.g., phenol and chloroform, so that the DNA enters the aqueous phase and the hydrolyzed products enter the organic phase. The DNA in the aqueous phase is then precipitated by the addition of alcohol. However, this organic extraction method is laborious and time consuming, and requires the use of phenol or other toxic organic solvents, and is therefore, a safety hazard.

In another approach, DNA is isolated by lysing the DNA source with a chaotropic substance, for example guanidinium salt, urea and sodium iodide, in the presence of a DNA binding solid phase. The released DNA is bound to a solid phase in a one step reaction, where the solid phase is washed to remove any residual contaminants. Although these methods have proven to be less time consuming and toxic, they still require pelleting and resuspending the cells to remove culture medium. Furthermore, lysing a nucleic acid source with a chaotropic substance is not compatible with some of the most common methods of nucleic acid isolation and purification, such as anion-exchange chromatography and Solid-phase Reversible Immobilization.

Once DNA is released from a cell, a number of methods exist for its purification. Purity is extremely important for sensitive downstream manipulations, such as automated sequencing.

One purification approach uses CsCl gradient centrifugation. CsCl gradient centrifugation uses the different sedimentation behaviors of differently sized nucleic acid molecules (RNA, plasmid DNA, genomic DNA) in a CsCl concentration gradient in the presence of intercalating agents, such as ethidium bromide, for the separation of nucleic acids. This type of separation can only be used with large quantities of nucleic acids and requires the use of ultracentrifuges. In addition to the high financial expenditure of an ultracentrifuge, a considerable expenditure of time (at least 48 hours) is required for double CsCl gradient purification, organic extraction to remove ethidium bromide, and dialysis to remove CsCl.

Some methods of purification, or combined lysis/purification, use toxic organic solvents (e.g., phenol and chloroform) to lyse bacterial culture for plasmid preparation. However, the phenol-chloroform method requires a tedious and time-consuming phase separation step. Further, working with toxic organic solvents is not desirable.

DNA and RNA are anions at neutral pH and can, therefore, be isolated by anion-exchange chromatography. The bacterial cells are typically lysed by alkaline lysis. The cellular proteins and genomic DNA are separated by means of detergents and subsequent centrifugation. The supernatant which contains the plasmid DNA is called the “cleared lysate.” The cleared lysate is applied over an anion exchange column, in which the desired RNA or DNA binds to the column, while impurities pass through.

In order to obtain good results, certain manipulations of the isolated nucleic acids require that the nucleic acids be sufficiently pure. One such manipulation involves automated sequencing. Typically, automated sequencing machines use nucleotides tagged with fluorochromes rather than radioactive nucleotides. Four different dyes are used, and when excited by a laser, they emit light at different wavelengths. The dyes can be used to label the primer or each of the four dideoxy chain terminators. Because each dideoxy reaction mixture is identified by a different label, the mixtures can be pooled and run on a single lane, rather than on the four separate lanes necessary with radioactive labeling. The fluorescently tagged dideoxy fragments migrate down the gel and pass through the beam of a laser where the fluorochromes are excited by the laser and emit light which is detected by a photomultiplier or CCD camera. Plasmids need to be sufficiently pure for automatic sequencing because sequencing is sensitive to impurities in nucleic acid preparations. Also, large numbers of different plasmids are usually prepared and used for sequencing, which gives rise to a need for a rapid and simple preparation.

Thus, there is still a need for a method of isolating and purifying nucleic acids that is efficient, quick, and requires less manipulation than current methods. Such a method should not require harvesting the cells containing the nucleic acids prior to lysis, and should be capable of automation. Moreover, it is desirable to produce sufficiently purified nucleic acids that can be used for further molecular manipulations, especially manipulations that are sensitive to impurities in nucleic acid preparations, such as automated sequencing, PCR, restriction digestion and subcloning.

### BRIEF DESCRIPTION OF THE DRAWINGS

FIG. 1 compares the amounts of plasmid DNA recovered with the use of an enzyme solution 1, comprising 50 mM Tris-HCl, pH 8.0, 200 mM EDTA, 5% Triton X-100 and 10 mg/ml RNase A (Tracks 3 and 5), and an enzyme solution 2, comprising 50 mM Tris-HCL, pH 8.0, 200 mM EDTA, 5% Triton X-100, 10 mg/ml RNase A and 20 mg/ml lysozyme (Tracks 4 and 6), at various incubation times. As can be seen, Tracks 4 and 6 display the greatest amounts of isolated and purified plasmid DNA, showing that utilizing an Extraction Enzyme Solution of the invention allowed plasmid DNA to be isolated and purified rapidly.

FIG. 2 compares the amounts of plasmid DNA recovered with the use of various extraction enzyme solution preparations. The comparison of various combinations of non-ionic detergent, lysozyme, ribonuclease and metal chelator shows that a synergistic effect exists not only for the use of all four components, together, but also, though to a lesser degree, for the use of lysozyme, non-ionic detergent and metal chelator on the amounts of isolated and purified plasmid DNA.

FIG. 3 is an electrophoresis gel showing that an Extraction Enzyme Solution of the invention can be combined with a binding solution for one-step plasmid isolation and purification. FIG. 3 additionally demonstrates that the amount of plasmid DNA recovered is dependent upon PEG concentration.

FIG. 4 shows a restriction digest of the plasmid pCMV-SPORT-βgal or the plasmid pCR II-TOPO prepared according to an embodiment of the invention and digested with both EcoR I and Xho I. FIG. 4 shows that plasmid DNA, isolated and purified with Extraction Enzyme Solutions of the invention, were readily digestible by restriction enzymes and could be utilized for high through-put clone screening and subcloning.

FIG. 5 is a typical chromatogram for pCMV-SPORT-βgal (in part) (SEQ. ID No. 1) after treatment with an Extraction Enzyme Solution of the invention. FIG. 5 shows that plasmid DNA, isolated and purified with Extraction Enzyme Solution of the invention could be used with automated fluorescent sequencing.

FIG. 6 is a typical chromatogram for pCR II-TOPO (in part) (SEQ. ID NO. 2) FIG. 6 shows that plasmid DNA, isolated and purified with Extraction Enzyme Solutions of the invention could be used with automated fluorescent sequencing.

### SUMMARY OF INVENTION

Claimed herein are methods, compositions and kits for rapidly isolating and purifying at least one nucleic acid directly from a biological sample, or for preparing a biological sample for subsequent isolation and purification of at least one nucleic acid, without prior removal of the biological sample's culture medium or cellular fluid (i.e., fluid containing cells or cellular materials). In more detail, claimed herein are methods, kits and compositions for isolating and purifying at least one nucleic acid directly from a biological sample, or for preparing a biological sample for subsequent isolation and purification of at least one nucleic acid, wherein the kits and compositions comprise an Extraction Enzyme Solution comprising a lysozyme, a ribonuclease, a metal chelator and a non-ionic detergent (hereinafter, the combinations of the four components are referred to as, “Extraction Enzyme Solution”). Further, some of the methods claimed herein utilize an Extraction Enzyme Solution. Other methods use a lysozyme for isolating and purifying at least one nucleic acid directly from culture, or for preparing a biological sample for subsequent isolation and purification of at least one nucleic acid.

The compositions and kits claimed herein can further comprise a binding solution. The methods can also utilize the compositions and kits with the binding solutions. Additionally, Extraction Enzyme Solutions allow for automation of the process of isolating and purifying nucleic acids.

Treatment of biological samples with an Extraction Enzyme Solution comprising a lysozyme, a ribonuclease, a metal chelator and a non-ionic detergent allows for a quick, efficient and cost-effective method to isolate and purify nucleic acids. With an Extraction Enzyme Solution, cells are not required to be harvested from the biological sample by centrifugation and the cell culture medium or cellular fluid need not be removed. This also eliminates the need to resuspend the cells from the pellet. Not to be bound by theory, the inventor believes that Extraction Enzyme Solutions allow for rapid enzymatic digestion in the cell culture or cellular fluid, thereby allowing for the manipulation of DNA/RNA after lysis, without any, or with a minimum of, interference from the culture medium component. Additionally, it is believed that, by utilizing an Extraction Enzyme Solution, RNA degradation by the ribonuclease is not as limited during cell lysis as it would be in the presence of a chaotropic salt.

The combination of all four components in an Extraction Enzyme Solution together shows effective cell lysis, RNA degradation and good recovery of nucleic acid. Additionally, an Extraction Enzyme Solution eliminates the cumbersome steps of cell harvesting and reconstitution, thereby simplifying and streamlining the process of nucleic acid isolation and purification. The invention described herein is not limited to combinations of the four components, although any component utilized alone or in combination with only one or two of the other components is not as effective as utilizing all four components together. Also, it has been found that utilizing lysozyme with a non-ionic detergent and/or a metal chelator (and not utilizing ribonuclease) is also effective, though to a lesser degree than utilizing all four components together. When ribonuclease is not utilized, the enzyme solution can be utilized for RNA and DNA extraction.

The Extraction Enzyme Solution of the processes, kits and compositions claimed herein are intended to be utilized on biological samples including, but not limited to, blood, saliva, tissues, cell cultures, cellular fluid, cellular materials and the like. To utilize the processes, kits and compositions claimed herein with blood and saliva, additional enzymatic components may need to be added, including, but not limited to proteases. Non-limiting examples of cell cultures include bacterial, plant, yeast and mammalian cell cultures. To utilize the processes, kits and compositions claimed herein with plant, yeast and mammalian cell cultures, additional enzymatic components may need to be added, including, but not limited to, cellulases and pectinases for plants, lyticases for yeast cells and proteases for mammalian cells.

The enzyme solutions claimed herein lyse cell cultures and release DNA rapidly; sufficient nucleic acids may be recovered in as little as about 10 seconds after treatment of the cell cultures with the enzyme solutions. However, one generally would wish to utilize the enzyme solutions for at least about one, two or five minutes. Favorable results should be achieved in less than about 15 minutes, or at least in less than about 30 minutes.

Nucleic acids isolated and purified by the processes, compositions and kits comprising the Extraction Enzyme Solution can be utilized for downstream applications such as, but not limited to, PCR, DNA sequencing, restriction digestion and subcloning.

### DETAILED DESCRIPTION OF INVENTION

One embodiment of the instant invention comprises a method for preparing a biological sample for subsequent isolation and purification of at least one nucleic acid, wherein said method comprises: 1) combining the biological sample, without prior removal of the biological sample's culture medium or cellular fluid, and an Extraction Enzyme Solution to form a lysate mixture, wherein the Extraction Enzyme Solution comprises a lysozyme, a ribonuclease, a metal chelator, and a non-ionic detergent; 2) incubating the lysate mixture; and 3) combining the mixture with a binding solution. Subsequent purification and isolation steps can then be carried out, including: 4) binding the nucleic acid to a solid support; and 5) eluting the nucleic acid from the solid support.

The Extraction Enzyme Solution causes cell lysis and RNA degradation, thereby allowing for isolation and purification of nucleic acids directly from culture without, or with minimal interference from the biological sample's culture medium or cellular fluid.

The Extraction Enzyme Solution can be utilized on biological samples including, but not limited to, blood, saliva, tissues, cell cultures, cellular fluid, cellular materials and the like. Non-limiting examples of cell cultures include bacterial, plant, yeast and mammalian cell cultures. Additional enzymatic components may need to be added when preparing biological samples containing plant, yeast or mammalian cells, including, but not limited to, cellulases and pectinases for plant cells, lyticases for yeast cells and proteases for mammalian cells. The processes claimed herein can be utilized for isolating and purifying nucleic acids, including DNA, whether circular or linear.

In the processes claimed herein, the user treats the biological sample briefly with the Extraction Enzyme Solution. The biological sample can be treated as briefly as about 10 seconds, although generally it should be treated for less than about 1 to less than about 2 minutes, although one of ordinary skill in the art would recognize that more or less time could also be effectively utilized (such as less than 5, 15, or 30 minutes).

Although an Extraction Enzyme Solution comprises a lysozyme, a ribonuclease, a metal chelator and a non-ionic detergent, additional components can also be utilized for stabilizing the enzyme solution for long term storage, including buffers, such as tris, and stabilizers, such as glycerol. The enzyme solution can also include multiple types of lysozymes, ribonucleases, metal chelators or non-ionic detergents.

The inventor believes that all lysozymes are useful in Extraction Enzyme Solutions. Concentrations of lysozyme can be as low as 0.5 mg/ml, or as high as 40 mg/ml, although concentrations of 10 and 30 mg/ml have been found to be effective. Similarly, it is believed that all ribonucleases are useful in the enzyme solution for rapid degradation of RNA. Concentrations of ribonuclease can be as low as 0.1 mg/ml, or as high as 20 mg/ml, although concentrations of 5 mg/ml and 10 mg/ml have been effective.

Metal chelators useful for practicing the instant invention include, but are not limited to, EDTA, EGTA, CDTA and combinations thereof. Concentrations of metal chelators can be as low as 10 mM, or as high as 300 mM, although concentrations of 100 and 200 mM have been effective.

Non-ionic detergents useful for practicing the instant invention include, but are not limited to polyoxyethylenes, alkylglucosides, alkylthioglucosides, and combinations thereof. Polyoxyethylenes include, but are not limited to, Triton X-100 (available from Sigma-Aldrich, and also known as polyethylene glycol tertoctylphenyl ether), Tween (available from Sigma-Aldrich, and also known as polyethylene glycol sorbitan monolaurate) and Igepal CA-630 (available from Sigma-Aldrich, and also known as (octylphenoxy)polyethylenglycol). Alkylthioglucosides include, but are not limited to octylthioglucosides, such as octyl-β-D-thioglucopyranoside (available from Sigma-Aldrich). Concentrations of non-ionic detergents can be as low as 0.5%, or as high as 10%, although concentrations of 1% and 5% are also effective.

The above process can also be utilized with a binding solution. The binding solution can be utilized after, or concurrently with Extraction Enzyme Solutions for subsequent capture of DNA to a matrix.

Various reagents can be utilized as the binding solution in conjunction with Extraction Enzyme Solutions. Binding solutions can include, without limitation, (1) alcohol or polyethylene glycol with or without salt, (2) combinations of alcohol, chaotrope and salt, and (3) combinations of polyethylene glycol, alcohol and salt.

Non-limiting examples of alcohol useful in the binding solution include isopropanol, ethanol, combinations thereof and the like. Non-limiting examples of salts useful in the binding solution include, but are not limited to sodium chloride, lithium chloride, potassium chloride, sodium acetate, potassium acetate, lithium acetate, combinations thereof and the like.

Non-limiting examples of chaotropes useful in the binding solution include guanidine thiocyanate, guanidine hydrochloride, sodium perchlorate, sodium iodine, combinations thereof and the like.

Brief centrifugation or vacuum filtration can be utilized to assist with DNA capture. DNA capture can be accomplished by chaotrope-driven binding mode or by precipitation-driven binding mode. Silica-based matrixes are essential for chaotrope-driven binding, but not essential for precipitation-driven binding.

After capture by means known to those skilled in the art (such as on a solid support), the nucleic acid (e.g., DNA) is washed with a wash solution to remove residual salts and other impurities. Bound plasmid DNA can then be selectively eluted by any known means, including, but not limited to, addition of low salt buffer or sterile distilled water. Non-limiting examples of wash solutions useful for removal of salts and other impurities from the bound nucleic acid include 60-80% ethanol and 50-70% isopropanol. Non-limiting examples of solutions useful to elute nucleic acids include 10 mM tris, pH 8.5 and sterile distilled water.

Following addition of the Extraction Enzyme Solution, DNA may be captured by means of anion exchanger or other surfaces. A number of surfaces have been well known to bind nucleic acids in certain conditions. These include, without limitation, silica dioxide, alumina oxide, diatomaceous earth, microparticles (such as carboxylated magnetic polystyrene beads and magnetic silica beads), and polymers (such as polyethylenimine). One may combine the Extraction Enzyme Solution with DNA binding microparticles or polymers to effect cell lysis and DNA capture at the same time.

An additional embodiment of the instant invention comprises a method for isolating and purifying at least one nucleic acid from a biological sample comprising combining a biological sample, without prior removal of the biological sample's cell culture medium or cellular fluid, with an Extraction Enzyme Solution and a binding solution to form a lysate mixture, wherein said Extraction Enzyme Solution comprises: 1) a lysozyme, 2) a ribonuclease, 3) a metal chelator, and 4) a non-ionic detergent; and wherein said binding solution comprises 1) polyethylene glycol and 2) salt; and incubating the lysate mixture for about 10 seconds to about 30 minutes. Subsequent nucleic acid isolation and purification steps can then be carried out including binding the nucleic acid to a solid support (such as beads, a porous matrix or other solid surfaces that have the proper functional groups) and eluting the nucleic acid from the solid support. The binding solution can be utilized after, or concurrently with the enzyme solution. The Extraction Enzyme Solutions and binding solutions useful for this embodiment, or for all embodiments claimed herein, are the same as described above.

An additional embodiment of the instant invention comprises a method of preparing bacterial cells for isolation and purification of at least one nucleic acid, wherein said method comprises adding an Extraction Enzyme Solution to a biological sample, without prior removal of the biological sample's culture medium or cellular fluid.

Yet another embodiment of the instant invention comprises an Extraction Enzyme Solution for preparing a biological sample for subsequent isolation and purification of at least one nucleic acid. The Extraction Enzyme Solution, as described above, comprises a lysozyme, a ribonuclease, a metal chelator and a non-ionic detergent.

The Extraction Enzyme Solution can comprise about 0.5 to about 40 mg/ml lysozyme, about 0.1 to about 20 mg/ml of ribonuclease, about 10 to about 300 mM metal chelator and about 0.5% to about 10% non-ionic detergent. The Extraction Enzyme Solution can additionally comprise glycerol and buffer. An additional embodiment of the Extraction Enzyme Solution comprises about 10 to about 30 mg/ml lysozyme, about 5 to about 10 mg/ml Ribonuclease A, about 100 to about 200 mM EDTA, about 1 to about 5% Triton X-100. Yet another embodiment of the Extraction Enzyme Solution comprises about 20 mg/ml lysozyme, about 10 mg/ml Ribonuclease A, about 200 mM EDTA and about 5% Triton X-100.

A further embodiment of the instant invention comprises an automated process for isolating and purifying a nucleic acid of interest. The automated process comprises: 1) means for cultivating cells, wherein said cells contain a nucleic acid of interest, 2) means for adding an Extraction Enzyme Solution to the cultivated cells, without prior removal of the cell culture medium, to form a lysate mixture, wherein said Extraction Enzyme Solution comprises: a) a lysozyme; b) a ribonuclease; c) a metal chelator; and d) a non-ionic detergent; 3) means for incubating the lysate mixture; 4) means for combining the lysate mixture with a binding solution, 5) means for binding the nucleic acid to a matrix, and 6) means for eluting the bound nucleic acid from the matrix.

Means for cultivating cells include, but are not limited to, growing cells (such as bacterial cells) on a selective solid medium, such as agar, then inoculating the cells into a liquid medium, such as LB broth, and allowing the cells to multiply overnight. Cell cultures can also be grown in high through-put vessels, such as multi-well plates and strip tubes. Alternatively, bacterial cell colonies can be scraped off of selective solid mediums and used directly for plasmid preparation.

Means for adding an Extraction Enzyme Solution to the cultivated cells include, but are not limited to pipettes, multi-channel pipettes, or adding a powder of a lyophilized form of an Extraction Enzyme Solution.

Means for combining the lysate mixture with a binding solution, include but are not limited to pipettes and mixing.

Means for binding the nucleic acid to a matrix include, but are not limited to the use of a matrix selected from the group consisting of: silica dioxide, alumina oxide, diatomaceous earth, microparticles (such as carboxylated magnetic polystyrene beads and magnetic silica beads), and polymers (such as polyethylenimine). The matrix can include combinations of the above materials. Means for eluting the bound nucleic acid from the matrix include, but are not limited to, the addition of low salt buffer or sterile distilled water.

The automated process is additionally useful, as it can be utilized without requiring a centrifuge to pellet the cells and without requiring removal of the culture medium.

A further embodiment of the instant invention is a kit for isolating and purifying at least one nucleic acid from a biological sample, or for preparing a biological sample for subsequent isolation and purification of at least one nucleic acid, without prior removal of the biological sample's cell culture medium or cellular fluid. The kit comprises an Extraction Enzyme Solution comprising: 1) a lysozyme, 2) a ribonuclease, 3) a metal chelator, and 4) a non-ionic detergent. The kit can also comprise a binding solution, which can be used with the Extraction Enzyme Solution, or after the Extraction Enzyme Solution has been utilized. Non-limiting examples of useful binding solutions include, but are not limited to: 1) alcohol or polyethylene glycol, 2) a combination of alcohol and salt. or 3) a combination of alcohol, salt, and/or chaotrope.

An embodiment of the kit comprises an Extraction Enzyme Solution comprising about 0.5 to about 40 mg/ml lysozyme, about 0.1 to about 20 mg/ml of ribonuclease, about 10 to about 300 mM metal chelator and about 0.5 % to about 10% non-ionic detergent. The kit's Extraction Enzyme Solution can additionally comprise glycerol and buffer. Yet another embodiment of the kit's Extraction Enzyme Solution comprises about 20 mg/ml lysozyme, about 10 mg/ml Ribonuclease A, about 200 mM EDTA, and about 5% Triton X-100.

A further embodiment of this invention comprises an enzyme solution for isolating and purifying at least one nucleic acid from a biological sample, or for preparing a biological sample for subsequent isolation and purification of at least one nucleic acid, without prior removal of the biological sample's cell culture medium or cellular fluid, wherein said enzyme solution comprises a lysozyme, and wherein said nucleic acid is DNA or RNA. Additional components of the enzyme solution can be selected from the group consisting of a non-ionic detergent, a metal chelator, combinations thereof and the like. This embodiment can also be utilized as a kit.

An additional embodiment of the instant invention comprises a process for isolating and purifying DNA comprising from a biological sample, or for preparing a biological sample for subsequent isolation and purification of at least one nucleic acid, comprising adding an enzyme solution comprising lysozyme to the biological sample, without prior removal of the biological sample's cell culture medium or cellular fluid. The enzyme solution can further comprise a non-ionic detergent, a metal chelator, combinations thereof, and the like. Without the addition of ribonuclease, these embodiments can be utilized not only for isolating and purifying DNA, but also for RNA. Binding solutions, as described above, can additionally be utilized in this process with, or after, the addition of the enzyme solution.

The advantageous properties of the invention described above can be observed by reference to the following examples, which illustrate embodiments, but do not limit, the invention.

### EXAMPLES

If not indicated specifically otherwise, all glass filter materials are available from Ahlstrom, while all other components and materials are available from Sigma-Aldrich.

### Example 1

This Example provides a general preparation of plasmid DNA directly from bacterial culture.

Step 1: Add Extraction Enzyme Solution comprising 50 mM Tris-HCl, pH 8.0, 200 mM EDTA, 5% Triton X-100, 10 mg/ml RNase A and 20 mg/ml Lysozyme (about 0.1 volume of the culture) to a bacterial culture in a tube, a well, or a DNA purification column and mix briefly for under 10 seconds by pipetting up and down, or inversion, or vortex. Incubate at room temperature for 1-2 minutes.

Step 2: Add binding solution (40% isopropanol, 1.8 M guanidine thiocyanate and 1.0 sodium chloride), about one volume of the culture, and mix briefly by pipetting up and down or inversion. Transfer the mixture into a DNA purification column and bind DNA to a matrix by centrifugation for 30 seconds at maximum speed or by vacuum filtration. The DNA binding column utilized for these experiments consisted of a plastic column (1.209 inches long, 0.508 inches in diameter on top and 0.165 inches in diameter on bottom), stuffed with layers of glass filter discs of various sizes.

Step 3: Wash the column with a wash solution (10 mM tris, pH 8.5, 80% ethanol) by centrifugation for 1 minute at maximum speed or by vacuum filtration.

Step 4: Elute plasmid DNA with a low salt buffer (10 mM tris, pH 8.5) or sterile distilled water by centrifugation for 30 seconds at maximum speed.

### Example 2

This Example isolates and purifies plasmid DNA directly from overnight culture, utilizing a variety of enzyme solutions, and shows the synergetic effects of utilizing an Extraction Enzyme Solution comprising a lysozyme, a ribonuclease, a metal chelator, and a non-ionic detergent on plasmid DNA recovery.

E. coli strain DH5α containing the plasmid pCMV-SPORTβgal (7.8 kb) was used for the plasmid preparation. In each case, a 200 μl aliquot of overnight culture in LB broth (OD₆₀₀=3.0) was loaded into a mini spin column packed with two layers of Ahlstrom glass filter paper Grade 121 and one layer of Ahlstrom glass filter paper Grade 151 (on bottom). To the overnight culture 20 μl of an enzyme solution was added and the mixture was incubated at room temperature for up to 2 minutes. The following enzyme solutions were used:

Enzyme Solution 1
50 mM Tris-HCl, pH 8.0,
200 mM EDTA,
5% Triton X-100,
10 mg/ml RNase A
Enzyme Solution 2
50 mM Tris-HCl, pH 8.0,
200 mM EDTA,
5% Triton X-100,
10 mg/ml RNase A,
20 mg/ml Lysozyme

After incubation, the lysate was mixed with 250 μl of binding solution (40% isopropanol, 6 M LiCl, 0.6 M guanidine thiocyanate) and the mixture was forced through the column by means of centrifugation at 14,000 rpm for 15 seconds. The column was then washed with 400 μl of wash solution (10 mM Tris-HCl, pH 8.0, 10 mM NaCl, 80% ethanol) by means of centrifugation at 14,000 rpm for 1 minute. Plasmid DNA was eluted from the column in 50 μl of elution solution (10 mM Tris, pH 8.5) by means of centrifugation at 14,000 rpm for 30 seconds.

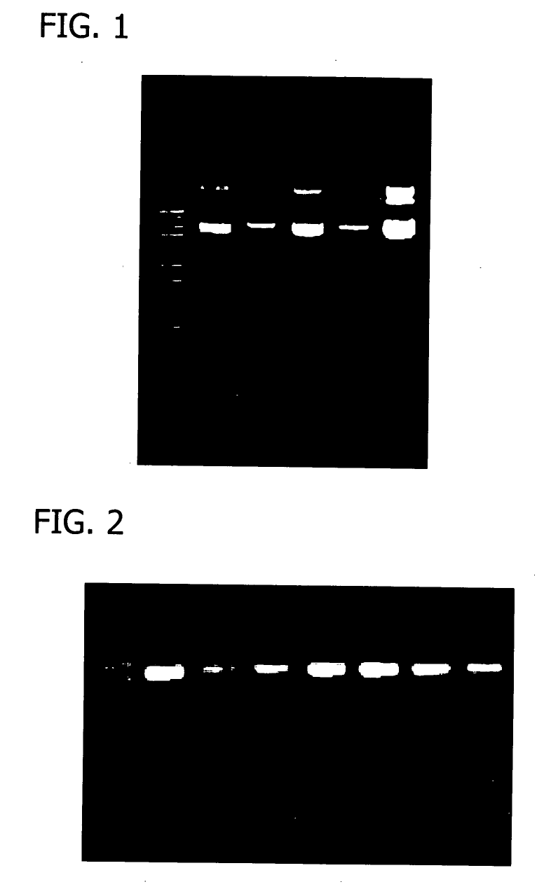

The amount of plasmid DNA isolated and purified in each preparation was determined with 5 μl of eluate by agarose gel electrophoresis. A 1 kb DNA ladder was loaded in Track 1 and 100 ng of control plasmid DNA purified by GenElute Endo-Free Maxi Kit was loaded in Track 2 (to provide a relative measurement of the amount of DNA in the gel). Samples treated with Enzyme Solution 1, with less than 1 minute of incubation time, were loaded in Track 3; samples treated with Enzyme Solution 2, with less than 1 minute of incubation time, were loaded in Track 4; samples loaded with Enzyme Solution 1, with 2 minutes of incubation time, were loaded in Track 5; and samples treated with Enzyme Solution 2, with 2 minutes of incubation time, were loaded in Track 6. The results of the gel of Example 2 are shown in FIG. 1.

### Example 3

This Example shows the synergetic effects of utilizing an Extraction Enzyme Solution comprising a lysozyme, a ribonuclease, a metal chelator, and a non-ionic detergent on plasmid DNA recovery.

A 350 μl aliquot of overnight culture in LB broth (OD₆₀₀=3.3) of E. coli DH5α containing the plasmid pCMV-SPORT-βgal was loaded into a mini spin column of the same type as in Example 2. The culture was then lysed with 35 μl of an enzyme solution for 2 minutes at room temperature. The following enzyme solutions were used:

Enzyme Solution 1
25 mM Sodium Acetate, pH 4.5,
10 mg/ml RNase A,
30 mg/ml Lysozyme
Enzyme Solution 2
25 mM Sodium Acetate, pH 4.5,
5% Triton X-100,
10 mg/ml RNase A,
30 mg/ml Lysozyme
Enzyme Solution 3
25 mM Sodium Acetate, pH 4.5,
5% Triton X-100,
200 mM EDTA,
10 mg/ml RNase A,
30 mg/ml Lysozyme
Enzyme Solution 4
50 mM Tris-HCl, pH 8.0,
5% Triton X-100,
200 mM EDTA,
10 mg/ml RNase A,
30 mg/ml Lysozyme
Enzyme Solution 5
50 mM Tris-HCl, pH 8.0,
200 mM EDTA,
10 mg/ml RNase A,
30 mg/ml Lysozyme
Enzyme Solution 6
50 mM Tris-HCl, pH 8.0,
5% Triton X-100,
10 mg/ml RNase A,
30 mg/ml Lysozyme

As can be seen, Enzyme Solutions 1, 2, 5 and 6 utilized less than all four components of the Extraction Enzyme Solution whereas Enzyme Solutions 3 and 4 utilized all four components.

The lysate was then mixed in each case with 350 μl of binding solution (40% isopropanol, 1 .8 M guanidine thiocyanate, 1 M NaCl) and the mixture was forced through the column by means of centrifugation. After a wash with 700 μl of an ethanol solution (10 mM Tris-HCl, pH 8.0, 10 mM NaCl), plasmid DNA was eluted in 50 μl of elution solution (10 mM Tris, pH 8.5).

Plasmid DNA recovery was determined by agarose gel electrophoresis with 1 μl of eluate in each case. A 1 kb DNA ladder was loaded in Track 1 and 100 ng control plasmid DNA purified by GenElute Endo-Free Maxi Kit was loaded in Track 2. Samples treated with Enzyme Solution 1 were loaded in Track 3; samples treated with Enzyme Solution 2 were loaded in Track 4; samples treated with Enzyme Solution 3 were loaded in Track 5; samples treated with Enzyme Solution 4 were loaded in Track 6; samples treated with Enzyme Solution 5 were loaded in Track 7; and samples treated with Enzyme Solution 6 were loaded in Track 8.

The results of Example 3 are shown in FIG. 2.

A comparison of the results of Example 2 and Example 3 additionally show that combinations of less than all four components also display favorable results, though to a lesser extent than utilizing all four components, so long as lysozyme is utilized. Thus, the combination of lysozyme and a metal chelator, and/or non-ionic detergent, also can be utilized for rapid extraction. This embodiment additionally can be utilized for RNA isolation and purification if a ribonuclease is not utilized.

### Example 4

This Example shows that the Extraction Enzyme Solutions of the instant invention may be utilized with binding solutions for plasmid DNA isolation.

A 350 μl aliquot of overnight culture in LB broth (OD₆₀₀=3.1) of E. coli DH5α containing the plasmid pCMV-SPORT-βgal was loaded into a mini spin column packed with three layers of Ahlstrom glass filter paper Grade 121. To the overnight culture 350 μl of a lysing/binding solution was added and the mixture was incubated at room temperature for 3 minutes. The following lysing/binding solutions were used:

Lysing/Binding Solution 1
10 mM Tris-HCl, pH 8.0,
40 mM EDTA,
1% Triton X-100,
2 mg/ml RNase A,
6 mg/ml Lysozyme,
8% PEG 8000,
0.6 M NaCl
Lysing/Binding Solution 2
10 mM Tris-HCl, pH 8.0,
40 mM EDTA,
1 mg/ml RNase A,
3 mg/ml lysozyme,
20% PEG 8000,
0.6 M NaCl

As can be seen, Lysing/Binding Solution 2 differs from Lysing/Binding Solution 1 by comprising a higher concentration of DNA binding agent PEG 8000.

After incubation, the mixture was forced through the column by means of centrifugation at 14,000 rpm for 30 seconds. The column was then washed with 500 μl of wash solution (10 mM Tris-HCl, pH 8.0, 10 mM NaCl) by means of centrifugation at 14,000 rpm for 1 minute. Plasmid DNA was eluted in 50 μl of elution solution (10 mM Tris, pH 8.5) by means of centrifugation at 14,000 rpm for 30 seconds.

Recovery of plasmid DNA was determined by agarose gel electrophoresis with 3 μl of eluate in each case. A 1 kb DNA ladder was loaded in Track 1 and 100 ng control plasmid DNA purified by GenElute Endo-Free Maxi Kit was loaded in Track 2. The samples treated with Lysing/Binding Solution 1 were loaded in Track 3 and the samples treated with Lysing/Binding Solution 2 were loaded in Track 4.

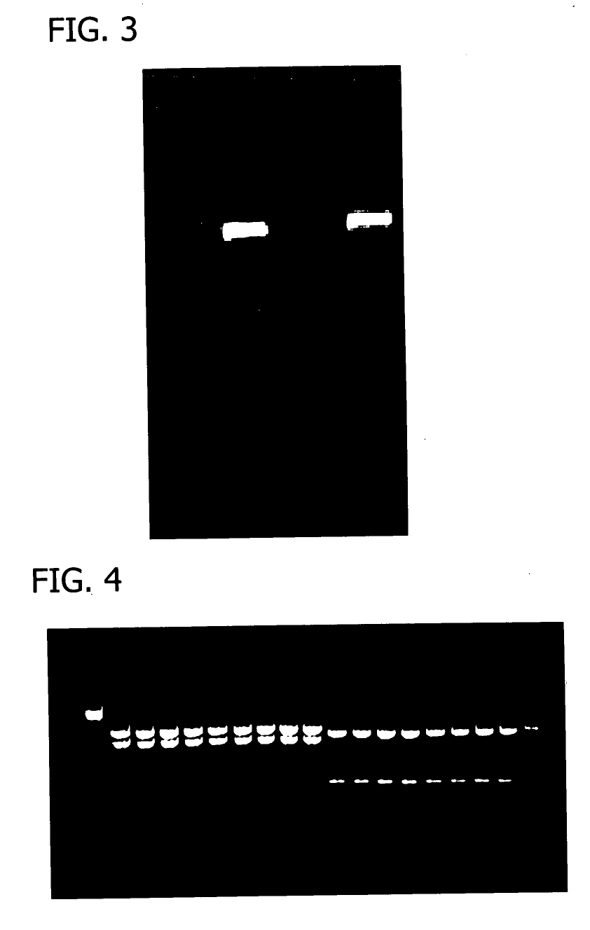

The results of Example 4 are shown in FIG. 3, and show that the amount of plasmid DNA recovered was dependent upon the level of DNA binding agent PEG 8000.

### Example 5

This Example shows that plasmid DNA, isolated and purified with Extraction Enzyme Solutions, is readily digestible by restriction enzymes and can be used with automated fluorescent sequencing.

Samples that contained the plasmid pCMV-SPORT-βgal in E. coli strain DH5α or the plasmid pCR II-TOPO with a 1.8 kb insert in E. coli strain TOPO 10 were used for plasmid preparation for restriction digestion and automated fluorescent sequencing. In each case, 400 μl of overnight culture in LB broth was treated for 2 minutes at room temperature with 40 μl of an enzyme solution (50 mM Tris-HCl, pH 8.0, 200 mM EDTA, 5% Triton X-100, 10 mg/ml RNase A, 20 mg/ml lysozyme). The lysate was then mixed with 400 μl of binding solution (40% isopropanol, 1.8 M guanidine thiocyanate, 1 M NaCl). By means of centrifugation or vacuum filtration, the mixture was forced through a mini spin column packed with one layer of Ahlstrom glass filter paper Grade 181 (on top) and two layers of Ahlstrom glass filter paper Grade 121. After the column was washed with an ethanol solution (10 mM Tris-HCl, pH 8.5, 80% ethanol), plasmid DNA was eluted in 40 μl of elution solution (10 mM Tris, pH 8.5).

For each restriction digestion, 2 μl of eluate containing the plasmid pCMV-SPORT-βgal or 3 μl of eluate containing the plasmid pCR II-TOPO with a 1.8 kb insert was digested with 3 units each of EcoR I and Xho I for 1 hr at 37° C. in a 15-μl reaction volume. The result is shown in FIG. 4.

A 1 kb DNA marker was loaded in Track 1. Uncut pCMV-SPORT-βgal was loaded in Track 2. Digests of pCMV-SPORT-βgal samples isolated by means of centrifugation were loaded in Tracks 3-6. Digests of pCMV-SPORT-βgal samples isolated by means of vacuum filtration were loaded in Tracks 7-10. Digests of pCMV-SPORT-βgal sample isolated by GenElute Plasmid Miniprep Kit were loaded in Track 11. Digests of pCR II-TOPO samples isolated by means of centrifugation were loaded in Track 12-15. Digests of pCR II-TOPO samples isolated by means of vacuum filtration were loaded in Tracks 16-19. Uncut pCR II-TOPO were loaded in Track 20.

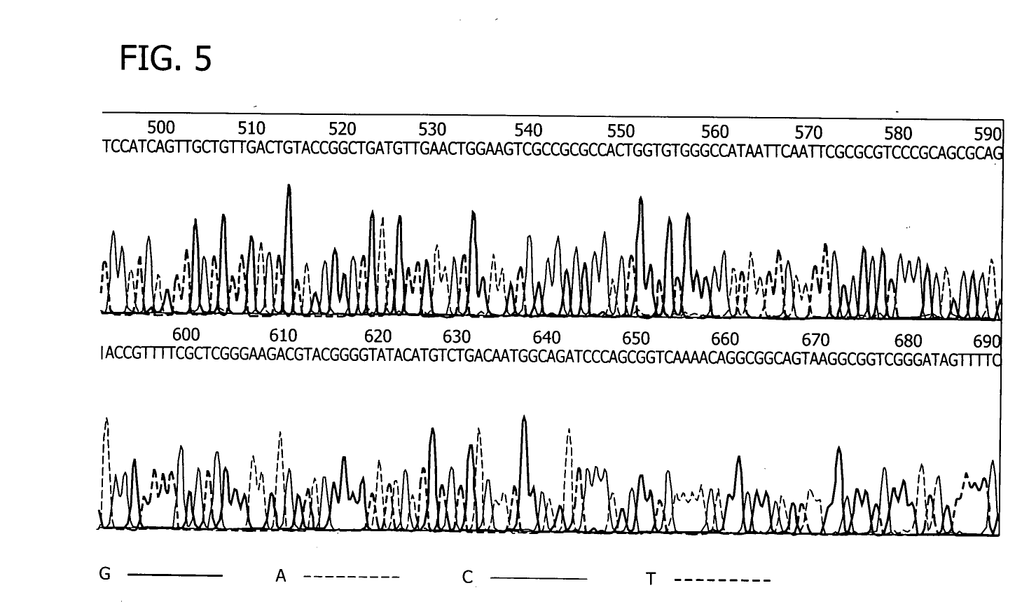

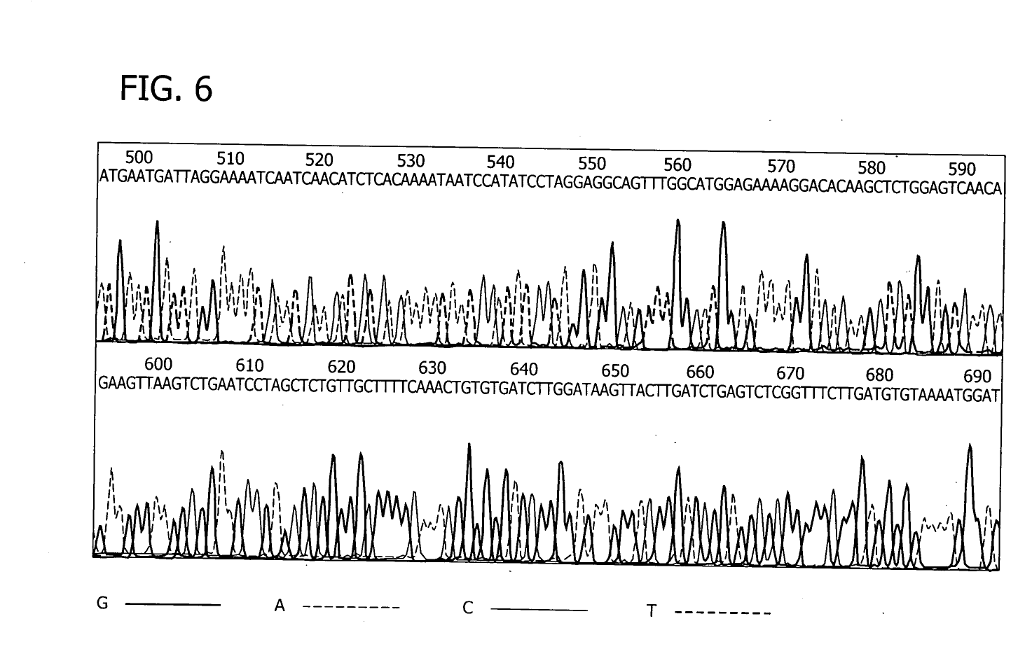

For automated fluorescent sequencing, 6 μl of eluate from each isolation by means of the said method was submitted to SeqWright (Houston, Tex.) for capillary sequencing with Big Dye 3.1. The averaged Phred 20 score of 8 independent preparations of pCMV-SPORT-βgal was 802±20. A typical chromatogram for pCMV-SPORT-βgal is shown in part in FIG. 5. The averaged Phred 20 score of 8 independent preparations of pCR II-TOPO was 768±50. A typical chromatogram for pCR II-TOPO is shown in part in FIG. 6.

Thus, it is apparent that there have been provided, in accordance with the instant invention, processes, compositions and kits that fully satisfy the objects and advantages set forth above. While the invention has been described with respect to various specific examples and embodiments thereof, it is understood that the invention is not limited thereto and many alternatives, modifications and variations will be apparent to those skilled in the art in light of the foregoing description. Accordingly, it is intended to embrace all such alternatives, modifications and variations as fall within the spirit and broad scope of the invention.

1. A method for preparing a biological sample for subsequent isolation and purification of at least one nucleic acid comprising:
a. combining the biological sample and an Extraction Enzyme Solution to form a lysate mixture, without prior removal of the biological sample's cell culture medium or cellular fluid, wherein the Extraction Enzyme Solution comprises: 1) a lysozyme, 2) a ribonuclease, 3) a metal chelator, and 4) a non-ionic detergent;
b. incubating the lysate mixture; and
c. adding a binding solution to the lysate mixture, wherein the binding solution is selected from the group consisting of: 1) an alcohol or a polyethylene glycol, 2) an alcohol and a salt mixture, or 3) an alcohol, salt, and/or chaotrope mixture.

2. The method of claim 1, wherein the biological sample is a cell culture.

3. The method of claim 2, wherein the cells in the cell culture are selected from the group consisting of bacterial, plant, yeast and mammalian.

4. The method of claim 1, wherein the lysate mixture is incubated for at least 10 seconds before the binding solution is added.

5. The method of claim 4, wherein the lysate mixture is incubated for less than 30 minutes before the binding solution is added.

6. The method of claim 5, wherein the lysate mixture is incubated for less than 5 minutes before the binding solution is added.

7. The method of claim 6, wherein the lysate mixture is incubated for less than 2 minutes before the binding solution is added.

8. The method of claim 1, further comprising washing the bound nucleic acid with a wash solution.

9. The method of claim 1, further comprising eluting the nucleic acid.

10. The method of claim 1, wherein the metal chelator is selected from the group consisting of EDTA, EGTA, CDTA and combinations thereof.

11. The method of claim 1, wherein the non-ionic detergent is selected from the group consisting of polyoxyethylenes, alkylglucosides, alkylthioglucosides, and combinations thereof.

12. The method of claim 11 wherein said alkylthioglucoside is octyl-β-D-thioglucopyranoside or wherein said polyoxyethylene is Triton X-100, Tween, or Igepal CA-630.

13. The method of claim 1, wherein said alcohol is selected from the group consisting of isopropanol, ethanol and combinations thereof.

14. The method of claim 1, wherein the salt is selected from the group consisting of sodium chloride, lithium chloride, potassium chloride, sodium acetate, potassium acetate, and lithium acetate.

15. The method of claim 1, wherein said chaotrope is selected from the group consisting of guanidine thiocyanate, guanidine hydrochloride, sodium perchlorate, sodium iodine and combinations thereof.

16. The method of claim 3, wherein the cells in the cell culture are yeast cells and the Extraction Enzyme Solution additionally comprises a lyticase.

17. The method of claim 3, wherein the cells in the cell culture are mammalian and the Extraction Enzyme Solution additionally comprises a protease.

18. The method of claim 1, wherein the nucleic acid is DNA.

19. The method of claim 1, further comprising isolating and purifying the nucleic acid by d) binding the nucleic acid to a solid support and e) eluting the bound nucleic acid from the solid support.

20. The method of claim 1, wherein the Extraction Enzyme Solution and binding solution are added at the same time.

21. A method for isolating and purifying at least one nucleic acid from a biological sample comprising:
a) combining a biological sample, without prior removal of the biological sample's cell culture medium or cellular fluid, with an Extraction Enzyme Solution and a binding solution to form a lysate/binding solution, wherein said Extraction Enzyme Solution comprises: 1) a lysozyme, 2) a ribonuclease, 3) a metal chelator, and 4) a non-ionic detergent; and wherein said binding solution comprises: 1) polyethylene glycol, and 2) salt, and
b) incubating the lysate/binding solution mixture for about 10 seconds to about 30 minutes.

22. The method of claim 21, wherein the biological sample is a cell culture.

23. The method of claim 22, wherein the cells in the cell culture are selected from the group consisting of bacterial, plant, yeast and mammalian cells.

24. The method of claim 21, further comprising c) binding the nucleic acid to a solid support and d) eluting the nucleic acid with a wash solution.

25. The method of claim 21, further comprising eluting the nucleic acid.

26. The method of claim 21, wherein the metal chelator is selected from the group consisting of EDTA, EGTA, CDTA, and combinations thereof.

27. The method of claim 21, wherein the non-ionic detergent is selected from the group consisting of polyoxyethylenes, alkylglucosides, alkylthioglucosides, and combinations thereof.

28. The method of claim 27, wherein said alkylthioglucoside is octyl-β-D-thioglucopyranoside or wherein said polyoxyethylene is Triton X-100, Tween, or Igepal CA-630.

29. The method of claim 21, wherein the lysate/binding solution is incubated for less than about 5 minutes.

30. The method of claim 21, wherein said nucleic acid is DNA.

31. The method of claim 21, wherein any or all steps are automated.

32. The method of claim 22, wherein the cells in the cell culture are yeast cells and the Extraction Enzyme Solution additionally comprises a lyticase.

33. The method of claim 22, wherein the cells in the cell culture are mammalian cells and the Extraction Enzyme Solution additionally comprises a protease.

34. A method for preparing a biological sample for subsequent isolation and purification of at least one nucleic acid, without prior removal of the biological sample's cell culture medium or cellular fluid, comprising adding an Extraction Enzyme Solution to a biological sample to form a lysate mixture, wherein said Extraction Enzyme Solution comprises:
a) a lysozyme;
b) a ribonuclease;
c) a metal chelator; and
d) a non-ionic detergent,
and allowing said lysate mixture to incubate for at least about 10 seconds to about 30 minutes, wherein said Extraction Enzyme Solution lyses said cells, thereby freeing said nucleic acid for isolation and purification.

35. An Extraction Enzyme Solution for rapidly isolating and purifying a nucleic acid from a biological sample, or for preparing a biological sample for subsequent isolation and purification of at least one nucleic acid, without prior removal of the biological sample's cell culture medium or cellular fluid, wherein said Extraction Enzyme Solution comprises:
a) a lysozyme;
b) a ribonuclease;
c) a metal chelator; and
d) a non-ionic detergent.

36. The Extraction Enzyme Solution of claim 35, wherein the biological sample is a cell culture.

37. The Extraction Enzyme Solution of claim 36, wherein the cells in the cell culture are selected from the group consisting of bacterial, plant, yeast and mammalian cells.

38. The Extraction Enzyme Solution of claim 35, wherein the metal chelator is selected from the group consisting of EDTA, EGTA, CDTA, and combinations thereof.

39. The Extraction Enzyme Solution of claim 35, wherein the non-ionic detergent is selected from the group consisting of polyoxyethylene, alkylglucosides, alkylthioglucosides, and combinations thereof.

40. The Extraction Enzyme Solution of claim 35, wherein the alkylthioglucoside is octyl-β-D-thioglucopyranoside or wherein said polyoxyethylene is Triton X-100, Tween, or Igepal CA-630.

41. The Extraction Enzyme Solution of claim 35, wherein said lysozyme is at a concentration of about 0.5 to about 40 mg/ml, said ribonuclease is at a concentration of about 0.1 to about 20 mg/ml, said metal chelator is at a concentration of about 10 to about 300 mM and said non-ionic detergent is at a concentration of about 0.5% to about 10%.

42. The Extraction Enzyme Solution of claim 35, additionally comprising a stabilizer and a buffer.

43. The Extraction Enzyme Solution of claim 42, comprising about 20 mg/ml lysozyme, about 10 mg/ml Ribonuclease A, about 200 mM EDTA, about 5% Triton X-100, about 20% glycerol and about 50 mM Tris-HCl, pH 8.0.

44. An automated process for isolating and purifying a nucleic acid of interest comprising:
a) cultivating bacterial cells, wherein said bacterial cells contain a nucleic acid of interest,
b) adding an Extraction Enzyme Solution to the cultivated bacterial cells, without prior removal of culture medium, to form a lysate mixture, wherein said Extraction Enzyme Solution comprises:
1. a lysozyme;
2. a ribonuclease;
3. a metal chelator; and
4. a non-ionic detergent
c) incubating the lysate mixture;
d) combining the lysate mixture with a binding solution;
e) binding the nucleic acid to a matrix; and
f) eluting the bound nucleic acid from the matrix.

45. A kit for isolating and purifying at least one nucleic acid from a biological sample, or for preparing a biological sample for subsequent isolation and purification of at least one nucleic acid, without removing the biological sample's cell culture medium or cellular fluid, wherein the kit comprises an Extraction Enzyme Solution comprising: 1) a lysozyme, 2) a ribonuclease, 3) a metal chelator, and 4) a non-ionic detergent.

46. The kit of claim 45, further comprising a binding solution.

47. The kit of claim 46, wherein said binding solution is selected from the group consisting of 1) alcohol or polyethylene glycol, 2) a combination of alcohol and salt, or 3) a combination of alcohol, salt, and/or chaotrope.

48. The kit of claim 46, wherein said binding solution can be utilized at the same time, or after, the Extraction Enzyme Solution.

49. A process for isolating and purifying at least one nucleic acid from a biological sample, or for preparing a biological sample for subsequent isolation and purification of at least one nucleic acid, wherein said nucleic acid is either RNA or DNA, comprising (1) adding an enzyme solution to a biological sample, without prior removal of the sample's cell culture medium or cellular fluid, to form a lysate mixture, wherein said enzyme solution comprises a lysozyme, and (2) allowing said lysate mixture to incubate for at least about 10 seconds to about 30 minutes.

50. The process of claim 49, wherein said enzyme solution further comprises at least one component selected from the group consisting of a non-ionic detergent, a metal chelator, and combinations thereof.

51. The process of claim 50, further comprising combining the lysate mixture with a binding solution, wherein the binding solution is selected from the group consisting of 1) alcohol or polyethylene glycol, 2) a combination of alcohol and salt, or 3) a combination of alcohol, salt, and/or chaotrope; and binding the nucleic acid to a solid support.

52. An enzyme solution for the rapid extraction of a nucleic acid, from a biological sample without removal of the biological sample's cell culture medium or cellular fluid, comprising a lysozyme.

53. The enzyme solution of claim 52, further comprising at least one component selected from the group consisting of a non-ionic detergent, a metal chelator and combinations thereof.

54. The enzyme solution of claim 53, further comprising a ribonuclease.

55. A kit for the rapid extraction of RNA or DNA from a biological sample, without removal of the biological sample's cell culture medium or cellular fluid, wherein said kit comprises a lysozyme.

56. The kit of claim 55, further comprising at least one component selected from the group consisting of a non-ionic detergent, a metal chelator and combinations thereof.

57. The kit of claim 56, further comprising a binding solution.

58. The kit of claim 57, wherein said binding solution is selected from the group consisting of 1) alcohol or polyethylene glycol, 2) a combination of alcohol and salt, or 3) a combination of alcohol, salt, and/or chaotrope.


SEQUENCE LISTING (2 sequences)

SEQ ID NO: 1 - DNA, 197 bp, Artificial Sequence
  Plasmid pCMV-SPORT-betagal
tccatcagttgctgttgactgtaccggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttc

SEQ ID NO: 2 - DNA, 197 bp, Artificial Sequence
  Plasmid pCRII-TOPO
atgaatgattaggaaaatcaatcaacatctcacaaaataatccatatcctaggaggcagtttggcatggagaaaaggacacaagctctggagtcaacagaagttaagtctgaatcctagctctgttgcttttcaaactgtgtgatcttggataagttacttgatctgagtctcggtttcttgatgtgtaaaatggat